(19) United States
(12) Patent Application Publication
(10) Pub. No.: US 2005/0209203 A1
(43) Pub. Date: Sep. 22, 2005

(54) Use of vitamin Ds or vitamin D analogs to treat cardiovascular disease

(76) Inventors: Jin Tian, Waukegan, IL (US); Joel Z. Melnick, Wilmette, IL (US); E. Scott Toner, Vernon Hills, IL (US); Jinshyun Ruth Wu-Wong, Libertyville, IL (US); David H. Ostrow, Lake Zurich, IL (US); Laura A. Williams, Gurnee, IL (US); Eugene Sun, Libertyville, IL (US); Dheerendra R. Kommala, Gurnee, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/002,934

(22) Filed: Dec. 2, 2004

Related U.S. Application Data
(63) Continuation-in-part of application No. 10/903,039, filed on Jul. 29, 2004.
(60) Provisional application No. 60/530,842, filed on Dec. 18, 2003.
(60) Provisional application No. 60/491,088, filed on Jul. 30, 2003.

(51) Int. Cl. A61K 31/59; A61K 31/401; A61K 9/70

(52) U.S. Cl. 514/167; 514/423; 424/449

(57) ABSTRACT

Disclosed are pharmaceutical compositions containing Vitamin D receptor activators or Vitamin D analogs to treat, prevent or inhibit vascular disease. The pharmaceutical compositions may also include ACE inhibitors or other agents. Also disclosed are methods of reducing PAI-1 expression by administering effective amounts of Vitamin D receptor activators or Vitamin D analogs to a mammal in need thereof. Additionally disclosed are methods of preventing, inhibiting or treating thrombosis in a mammal in need of such prevention, inhibition or treatment comprising administering effective amounts of Vitamin D receptor activators or Vitamin D analogs to the mammal.

### CROSS REFERENCE TO RELATED APPLICATIONS

The subject application is a Continuation-In-Part of, and claims priority to, pending U.S. Patent Application Ser. No. 10/903,039, filed on Jul. 29, 2004, which claims priority to abandoned U.S. Provisional Application No. 60/491,088, filed on Jul. 30, 2003. The present application also claims priority to pending U.S. Provisional Application No. 60/530,842, filed on Dec. 18, 2003. All of the cited applications are hereby incorporated in their entirety by reference.

### FIELD OF THE INVENTION

The present invention relates to the use of a Vitamin D receptor activator (VDRA), preferably paricalcitol, or a Vitamin D analog, to treat and prevent cardiovascular disease, including cerebrovascular and peripheral vascular diseases, especially heart failure, cardiomyopathy, atherosclerosis, myocardial infarction, and cerebrovascular accidents.

### BACKGROUND OF THE INVENTION

Complications of cardiovascular diseases (CVD) due to atherosclerosis and cardiomyopathy are the most common cause of death in Western societies. Hypertension and hyperlipidemia in particular are major cardiac risk factors. Certain medications that treat hypertension (e.g., angiotensin converting enzyme inhibitors (ACEIs)) and abnormal lipid levels have been proven to reduce cardiovascular mortality significantly in high-risk populations such as hemodialysis patients. However, several factors, including adverse side effects, limit the utility of existing medications for preventing progression of cardiovascular disease or otherwise render these medications inadequate for treatment of CVD, particularly critical for high-risk populations.

The biological effects of VDRAs are mediated by the vitamin D receptor (VDR), a member of the superfamily of nuclear hormone receptors. One mechanism by which the VDR is believed to mediate biological effects is through activation of a transcription factor that binds to specific DNA sequence elements in vitamin D responsive genes and ultimately influences the rate of RNA polymerase II mediated transcription. VDRs are present in most human cell types, especially in the cardiovascular system and immune system.

Several lines of evidence support the idea that vitamin D plays an important role in the regulation of cardiovascular physiology as described in FIG. 1. Vitamin D has the potential to prevent atherosclerosis and vascular calcification through its effects on the immune system to down-regulate inflammatory pathways and to restore the normal expression of inhibitors of vascular calcification. Vitamin D also effects cell proliferation. Low vitamin D levels were associated with congestive heart failure. Vitamin D has direct effects to antagonize endothelin-1 induced cardiomyocyte hypertrophy. Finally, VDRAs down-regulate RAAS by inhibiting renin synthesis. Thus, treatment with VDRAs/vitamin D analogs may prevent or treat cardiovascular disease by affecting one or all of the pathways in FIG. 1.

However, in vitro and animal data have suggested that VDRAs and/or vitamin D analogs can damage the heart in uremia patients, for example, by causing vascular calcification, myocardial infarction, heart failure, cardiomyopathy and cerebrovascular accidents. Therefore, the medical community does not endorse use of these compounds as a therapy for cardiovascular disease and recommends the limitation of their use.

### SUMMARY OF THE INVENTION

The present invention is directed to methods for preventing, treating and delaying progression of vascular diseases, including cardiovascular, cerebrovascular and peripheral vascular diseases, especially heart failure, cardiomyopathy, atherosclerosis, myocardial infarction, and cerebrovascular accidents and pharmaceutical compositions useful therefor.

According to one embodiment, the present invention relates to VDRAs or Vitamin D analogs (referred to herein as “VDRA/Vitamin D analog”)-containing compositions for preventing, treating and delaying progression of vascular disease According to some aspects of the present invention, Vitamin D receptor activator (VDRA) compounds can be used. VDRAs include paricalcitol, calcitriol, 22-oxa-1-alpha,25-dihydroxyvitaminD2, MC-903 (calcipotriol), 16-ene-23-yne-1alpha, 25-dihydroxyvitamin D3, and 24-difluoro-26,27-dimethyl-16-ene-1alpha, 25-dihydroxyvitamin D3 (described in greater detail by DeLuca, et al., in PNAS, 2004, vol. 101, No.18, p. 6900-6904, incorporated herein by reference), compounds listed in Table 1 of Physiol.Rev. October 1998, Vol. 78, No. 4, p1193-1231, incorporated herein by reference in its entirety, and the so-called Gemini compounds (described in greater detail by Maehr, et al. in J. Steroid Biochem. Mole. Biol. 8990, 2004, 35-38, incorporated herein by reference), EB-1089 (a LEO Pharmaceuticals compound), and ED-71 (a Roche compound). Paricalcitol is especially preferred since it is a selective VDRA. Paricalcitol is commercially available from Abbott Laboratories (North Chicago, Ill., under the tradename ZEMPLAR).

According to other aspects of the present invention, the Vitamin D analog can be doxercalciferol or alfacalcidol.

According to some embodiments, especially preferred compositions of the present invention also include one or more of the following agents: an angiotensin converting enzyme inhibitor (ACEI) or an angiotensin II receptor 1(AT1) blocker or an aldosterone blocker (ARB). Compositions according to the present invention can also include other agents used to treat or prevent cardiovascular disease, such as beta blockers, calcium channel blockers, antilipemic agents, antihypertensive agents and antiinflammatory agents, including aspirin.

According to some aspects of the invention, pharmaceutical compositions can be administered through a sustained (or continuous) delivery system. The present invention also contemplates other modes of administration, including but not limited to oral, injectable and transdermal.

The present invention also includes a method of treating, inhibiting or preventing thrombosis in a mammal in need of such treatment, inhibition or prevention, comprising the step of administering to the mammal an effective amount of a Vitamin D receptor activator or Vitamin D analog. The Vitamin D receptor activator may be, for example, paricalcitol or calcitriol, and the Vitamin D analog may be, for example, doxercalciferol or alfacalcidol.

All patents and publications referred to herein are hereby incorporated in their entirety by reference.

### BRIEF DESCRIPTION OF THE FIGURES

FIG. 1 schematically represents the role of Vitamin D in deregulation of various inflammatory factors associated with atherosclerosis and its association with cardiomyocyte remodeling.

FIG. 2 presents bar graphs comparing median hospitalizations per year and hospital days per year for paricalcitol, calcitriol and no D therapy.

FIG. 3 presents bar graphs comparing results of regression analysis showing treatment with paricalcitol was associated with fewer hospitalizations and hospital days per year compared to no D.

FIG. 4 illustrates a Northern blot which evidences that paricalcitol treatment of As4.1-hVDR cells dose-dependently inhibits renin mRNA expression.

FIG. 5 illustrates the results of a renin promoter-luciferase assay used to examine the activity of paricalcitol to suppress renin gene transcription.

FIG. 6 illustrates the effect of paricalcitol and calcitriol on PAI-1 in primary culture of human coronary artery smooth muscle cells.

FIG. 7 illustrates the effect of vitamin D analogues on expression of the NPR-A gene promoter. VD3 represents 1,25 dihydroxyvitamin D (all results are normalized for co-transfected CMV Renilla luciferase expression).

FIG. 8 shows the effect of vitamin D analogues on ANP-stimulated cyclic GMP accumulation where ANP-dependent cGMP generation was used as a surrogate for ANP activity.

FIG. 9 shows the effect of vitamin D analogues on mutant (VDRE-deleted) NPR-A gene promoter in neonatal rat aortic smooth muscle cells; results are normalized for Renilla luciferase expression. Results suggest that all tested compounds induce ANP through the vitamin D response element.

FIG. 10 shows the effect of vitamin D analogues on basal vs. endothelin (10⁻⁷ M) stimulated hBNP gene promoter activity using transfected cardiac myocytes that were cultured in serum free medium.

FIG. 11 shows the effect of vitamin D analogues on basal and endothelin (10⁻⁷ M) stimulated hBNP gene promoter activity using transfected cardiac myocytes cultured in 0.2% fetal bovine serum. All cells were co-transfected with expression vectors directing expression of hVDR and hRXR.

FIG. 12 shows the effect of vitamin D analogues on basal and endothelin (10⁻⁷ M) stimulated Cdk2 activity in neonatal rat aortic smooth muscle cells.

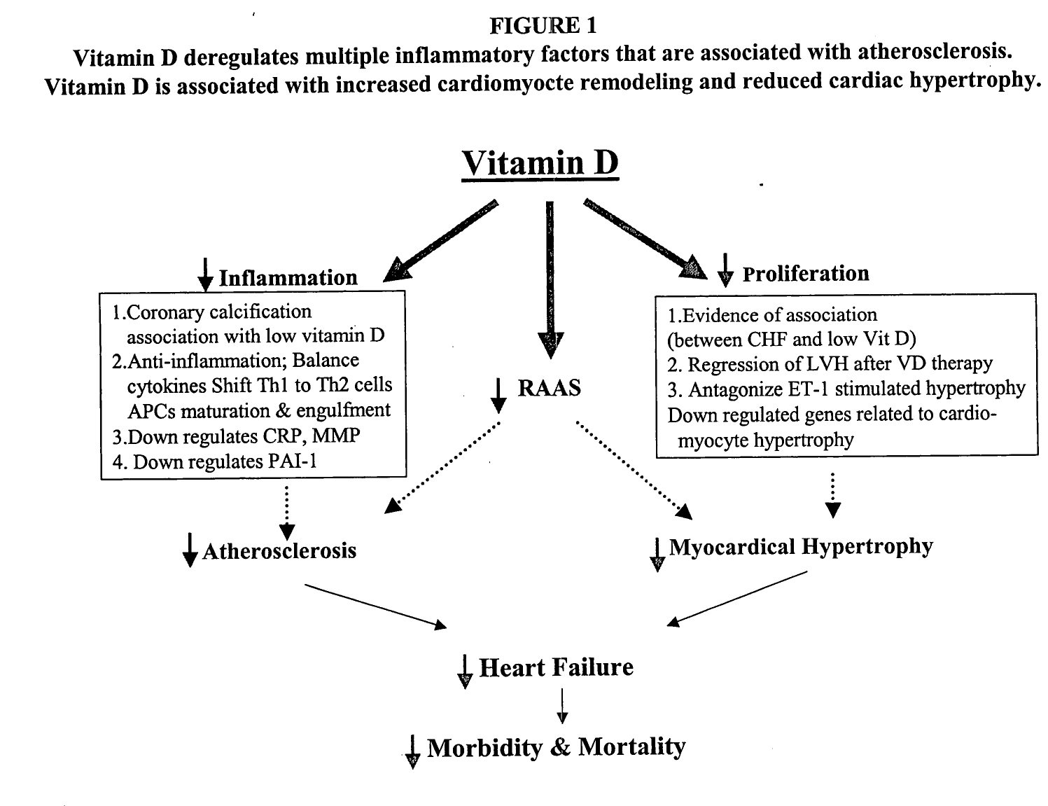

### DESCRIPTION OF THE EMBODIMENTS OF THE INVENTION

The present invention is generally directed to compositions containing a VDRA/vitamin D analog to treat or prevent cardiovascular diseases (CVD), including cardiomyopathy, coronary arterial, cerebrovascular and peripheral vascular diseases. The present invention also relates to methods of treating CVD by administering to a patient a pharmaceutical composition, which may be a sustained release formulation, containing a therapeutically effective amount of a VDRA/Vitamin D analog.

Treatment of patients with CVD by administration of a therapeutically effective amount of a VDRA/Vitamin D analog-containing composition is expected to be advantageous for effective reduction of renin expression, decreased inflammation and improved cardiac function directly through the therapeutic action of the VDRA/Vitamin D analog on cardiac tissue. In contrast, conventional treatments based on administration of an ACEI (i.e., without a VDRA/Vitamin D analog) for example, only reduce angiotensin (II), but do not reduce renin levels or act on Vitamin D receptors in the heart, vasculature and immune system itself. Administration of ACEI may not be an attractive long term treatment due to adverse consequences.

According to some aspects of the present invention, the inventive compositions contain a VDRA/vitamin D analog and at least one of the following agents: an ACE inhibitor, an angiotensin (II) receptor blocker (ARB) and aldosterone blocker in therapeutically effective amounts to inhibit renin production or inhibit activation of the renin-angiotensin-aldosterone system. Preferred compositions contain paricalcitol with at least one of these other agents. Such combinations can avoid ACE inhibition escape and aldosterone escape with subsequent increase in angiotensin (II) and aldosterone generation

Suitable ACE inhibitors, ARB and aldosterone blockers are commercially available. Suitable ACE inhibitors include, but are not limited to: captopril (commercially available under the tradename CAPOTEN from Mylan), enalapril (commercially available under the tradename VASOTEC from Merck), fosinapril (commercially available under the tradename MONOPRIL from Bristol Myers Squibb), benzapril (commercially available under the tradename LOTENSIN from Novartis Pharmaceuticals), moexipril (commercially available under the tradename UNIVASC from Schwarz Pharma), perindopril (commercially available under the tradename ACEON from Solvay), quinapril (commercially available under the tradename ACCUPRIL from Parke-Davis), ramipril (commercially available under the tradename ALTACE from Monarch), trandolapril (commercially available under the tradename MAVIK from Abbott Laboratories of North Chicago, Ill.), lisinopril (commercially available under the tradenames PRINIVIL from and ZESTRIL from Astra Zeneca).

Suitable angiotensin receptor blocking agents include, but are not limited to: losartan (commercially available as COZAAR from Merck), irbesartan (commercially available as AVAPRO from Bristol Myers Squibb and Sanofi), candesartan (commercially available as ATACAND from Astra Zeneca), eprosartan (commercially available as TEVETEN from Biovail Corporation of Canada), telmisartan (commercially available as MICARDIS from Boehringer Ingelheim) and valsartan (commercially available as DIOVAN from Novartis).

Suitable aldosterone blockers include, but are not limited to: eplerenone (commercially available under the tradename INSPRA from Pharmacia ), spironolactone (commercially available under the tradenames Aldactone, Adultmin, Aldopur, Aldospirone, Almatol, Berlactone, Diatensec, Diram, Esekon, Hypazon, Idrolattone, Merabis, Novospiroton, Osiren, Osyrol, Pirolacton, Resacton, Sincomen, Spiractin, Spiroctan, Spirolacton, Spirolang, Spironex, Spirotone, Tevaspirone, Verospiron, Xenalon Lactabs, Youlactone).

Additional components, e.g., physiologically acceptable carriers, solvents, binders, antioxidants, colorants, substrates can be used as necessary or desired.

Preferred treatment or preventative regimens for patients with CVD according to the present invention would administer therapeutically effective VDRA/Vitamin D analog-containing compositions according to the invention for a sufficient period to effect sustained or continuous delivery. As used herein, a “therapeutically effective dose” is a dose which in susceptible subjects is sufficient to prevent progression or cause regression of CVD or which is capable of relieving the symptoms caused by CVD.

An exemplary dosing regimen would provide the equivalent of about 0.5 micrograms of calcitriol per day or at least about 1 microgram calcitriol by injection three times weekly. For paricalcitol, a suitable dosing regimen would provide the equivalent of about 2 micrograms paricalcitol daily or at least about 4 micrograms paricalcitol three times weekly administered as a bolus. Suitable dosing regimens for other VDRA/Vitamin D analogs, e.g., doxercalciferol, can be determined straightforwardly by those skilled in the art based on the therapeutic efficacy of the VDRA/Vitamin D analog to be administered.

Since ACEI, ARB and aldosterone inhibitors have different efficacies and affect the body through different pathways than Vitamin D does, compositions according to the present invention can incorporate an ACEI, ARB or aldosterone inhibitor to be administered according to conventional dosing regimens, which are well known and readily available to those skilled in the art.

The invention also contemplates continuous or sustained drug delivery forms containing the selected VDRA/Vitamin D analog, and an ACEI and/or an ARB and/or an aldosterone blocker. Suitable delivery forms include, but are not limited to, tablets or capsules for oral administration, injections, transdermal patches for topical administration (e.g., drug to be delivered is mixed with polymer matrix adhered to or absorbed on a support or backing substrate, e.g., ethylcellulose), depots (e.g., injectable microspheres containing the desired bioactive compounds) and implants. Techniques for making these drug delivery forms are well known to those skilled in the art.

Further, it should also be noted that CKD patients undergoing hemodialysis often require the formation of an arteriovenous (A-V) fistula for hemodialysis (HD). The autogenous A-V fistula has long been proven to be the most durable access for HD. Primary failure of vascular access is mainly related to thrombosis. The pathophysiology underlying stenosis formation is turbulenceof blood flow, which activates platelets and endothelialcells. The final trigger causing thrombosis is a critical reduction of fistula blood flow. In this context, a particular role has been postulated for platelet-derived growth factor (PDGF)

Based upon the data presented in Example 5 below, it can be concluded that there is a statistically significant association with Zemplar therapy and fewer vascular access changes. Thus, Zemplar may have a beneficial effect through its action on endothelial cells, platelets and PDGF which are responsible for thrombosis. Future studies should clarify the mechanism of the proposed effect, understand if it extends beyond AV fistulas to grafts, dose-time dependency and the association with CaxP Product.

The present invention may be illustrated by the used of the following, non-limiting examples:

### EXAMPLE 1

### Decreased Morbidity and Mortality Associated With Vitamin D Therapy

The leading cause of mortality and morbidity in patients receiving chronic hemodialysis related to cardiovascular disease. Prevalence of CVD can be found in at least 75% of patients who initiate hemodialysis therapy.

An observational cohort study examining hemodialysis patients who started vitamin D therapy with paricalcitol experienced fewer hospitalizations related to cardiovascular events and non-infectious inflammations, compared with patients treated with calcitriol (Paricalcitol-treatedpatients experience improved hospitalization outcomes compared with calcitriol-treated patients in real-world clinical settings, D. G. Dobrez, et al. Nephrol Dialysis Transplant 2004 19:1174).

This study was expanded to include patients who received no Vitamin D receptor activator treatment. [“Improved hospitalization outcomes in hemodialysis patients treated with paricalcitol.” J. Melnick, et al., abstract book from World Congress of Nephrology, June 8-12, 2003, Berlin. Page 148] revealed that paricalcitol treatment was associated with improved hospitalization outcomes in hemodialysis (HD) patients who were treated with paricalcitol or with calcitriol compared to patients who did not receive any vitamin D treatment.

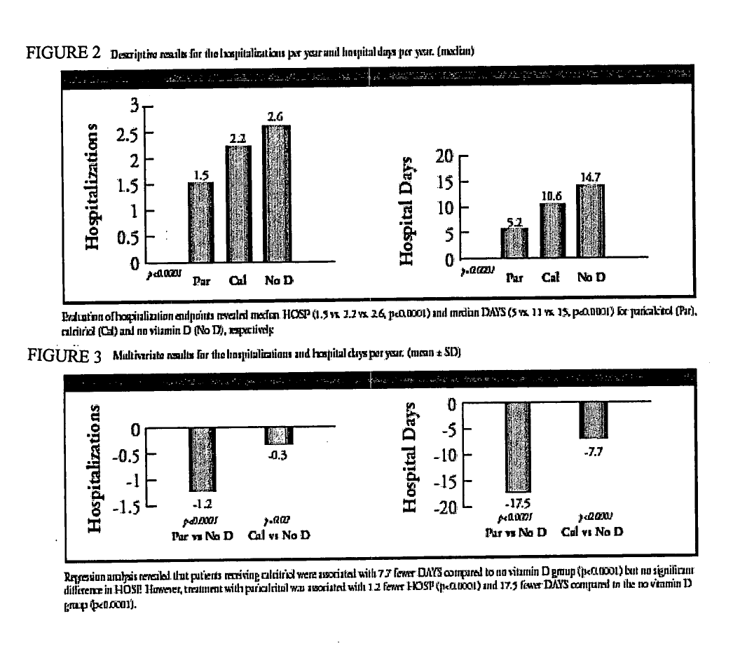

As shown in FIG. 2, evaluation of hospitalization endpoints revealed the median number of hospitalizations in a year for patients receiving a VDRA (either paricalcitol (“Par”) or calcitriol (“Cal”)) was lower than for patients who received no Vitamin D (“No D”). Notably, hospitalizations were fewer for patients treated with paricalcitol (1.5) than for those treated with calcitriol (2.2). In addition, the median number of days spent in the hospital was lower for patients receiving a VDRA (especially paricalcitol) compared to patients who received no Vitamin D (2.6). The number of hospital days was again lowest for paricalcitol (5.2) compared to calcitriol (10.6) and no Vitamin D (14.7).

FIG. 3 presents multivariate results for the hospitalizations and hospital days per year. Regression analysis of this data revealed receiving calcitriol was associated with 7.7 fewer hospitalization days compared to the No Vitamin D group, even though there was no statistical difference in the number of hospitalizations. However, treatment with paricalcitol was associated with 1.2 fewer hospitalizations and 17.5 fewer hospital days compared to the No Vitamin D group.

### EXAMPLE 2

### Activity of Paricalcitol to Suppress Renin Expression

Recently, it has been found that 1,25-dihydroxyvitamin D functions as a negative regulator of renin biosynthesis in vitro and in in vivo studies. Calcitriol is able to inhibit renin gene expression, which provides a molecular basis to explore the use of vitamin D and vitamin D analogs as new renin inhibitor to regulate rennin-angiotensin-aldosterone system RAAS).

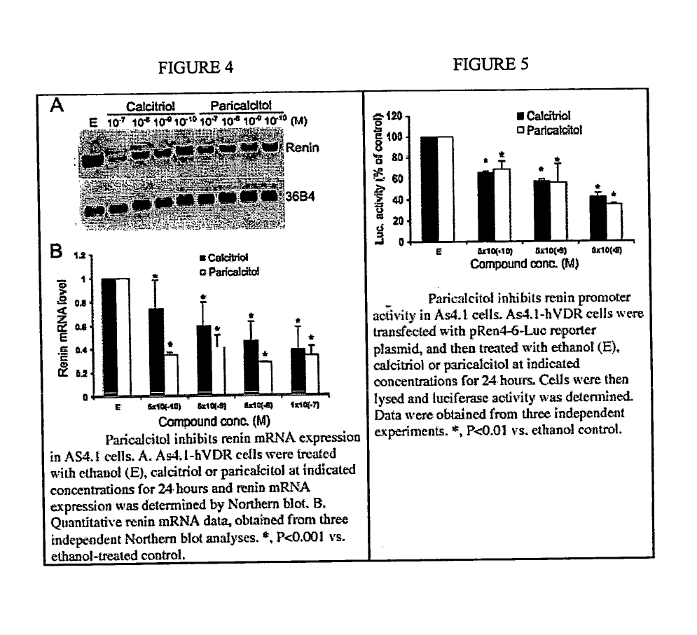

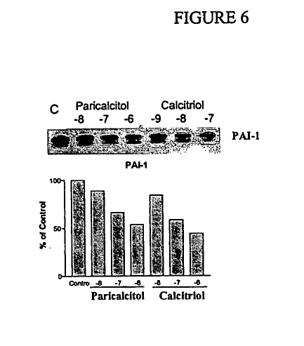

Using an in vitro cell culture system, the activity of paricalcitol to suppress renin gene expression was examined using previously published techniques (1,25-Dihydroxyvitamin D₃ is a negative endocrine regulator of the renin-angiotensin system, J. Clin. Invest., July 2002). As shown in FIG. 4, by Northern blot analysis, paricalcitol treatment of As4.1-hVDR cells d25e-dependently inhibits renin mRNA expression. In fact, its renin-inhibiting activity appears a bit more potent than calcitriol (FIGS. 4A and B). This inhibitory effect is confirmed by renin promoter-luciferase reporter assays, which examine the activity of paricalcitol to suppress renin gene transcription. In these assays, paricalcitol appears at least as potent as calcitriol to suppressing the activity of the renin gene promoter (FIG. 6).

This data supports the utility of a VDRA/Vitamin D analog to regulate the renin-angiotensin-tensin-aldosterone system and its criticality in CVD development and delay in progression of cardiovascular disease.

### EXAMPLE 3

### Effect of VDR Activators on PAI-1

The effect of paricalcitol and calcitriol on PAI-1 in primary culture of human coronary artery smooth muscle cells was investigated. (See FIG. 6.) PAI-1 (plasminogen activator inhibitor type-1) is one of the risk markers for coronary heart disease, and is enhanced in atherosclerotic plague and colocalized with macrophages.

Human coronary artery smooth muscle cells were incubated with paricalcitol or calcitriol at the indicated concentration for 24 hr at 37° C. Samples were solubilized in SDS-PAGE sample buffer, and the protein content in each sample was determined by the Bio-Rad dye-binding protein assay. Samples were resolved by SDS-PAGE using a 4-12% gel, and proteins were electrophoretically transferred to PVDF membrane for Western blotting. The membrane was blotted for 1 h at 25° C. with 5% nonfat dry milk in PBS-T and then incubated with a mouse anti-PAI-1 monoclonal antibody in PBS-T overnight at 4° C. The membrane was washed with PBS-T and incubated with a horseradish peroxidase-labeled anti-rabbit antibody for 1 h at 25° C. The membrane was then incubated with detection reagent (SuperSignal WestPico). The specific bands were visualized by exposing the paper to Kodak BioMax films.

FIG. 6 shows the results from Western blot using an anti-PAI-1 antibody. Two observations may be noted in these studies: (1) 100% inhibition of growth was never achieved even at 1 μM of any of the test compound. Confocal microscopy studies confirm that, although these drugs are potent in inducing the translocation of VDR from cytoplasm to nucleus, not all cells respond to VDRAs even after 2 h of exposure, which may explain the <100% inhibition. (2) Although paricalcitol is known to be less potent than calcitriol in the clinical studies, it exhibits similar potency to calcitriol in this assay. By checking the effect of drugs on the expression of 24(OH)ase, it was found that paricalcitol is less potent than calcitriol on stimulating the expression of 24(OH)ase, which may partially explain the higher potency of paricalcitol in this assay. These results show that paricalcitol and calcitriol are equally potent in reducing the PAI level in human coronary artery smooth muscle cells. Paricalcitol is usually dosed approximately 4 fold higher than calcitriol in the clinical situation, which may translate into a 4 fold higher potency in regulating the function of smooth muscle cells.

### EXAMPLE 4

### Effect of Paricalcitol in in vitro Models Using Myocardial or Vascular Smooth Muscle Cells in Culture

Experimentally induced vitamin D deficiency is associated with cardiac hypertrophy and hypertension in otherwise normal adult Sprague-Dawley rats (Weishaar et al., Am. J. Physiol. 1990 Jan; 258 (1 Pt 1):E13442). Cardiac hypertrophy is also seen in the VDR −/− mouse (Li et al., J. Clin. Invest. 2002 Jul; 110 (2):229-38), although this occurs in the setting of a 10-15 mm Hg elevation in systolic blood pressure implying that the hypertrophy may, as least in part, reflect increased ventricular overload. Vitamin D has been shown to inhibit endothelin (ET)-induced hypertrophy of neonatal rat cardiac myocytes in culture (Wu et al., J. Clin. Invest. 1996 Apr 1; 97(7):1577-88 and Li et al., J. Biol. Chem. 1994 Feb 18; 269(7):4934-9). This is associated with a reduction in expression of the ANP, BNP and α skeletal actin genes and suppression of the human ANP and BNP gene promoters (Wu et al., Am. J. Physiol. 1995 Jun; 268 (6 Pt 1):E1108-13.

In the present study, we considered whether paricalcitol possesses similar effects (vs. the native hormone) in several in vitro models using myocardial or vascular smooth muscle cells in culture.

Effect of VDRA/Vitamin D Analogs on NPR-A Gene Promoter Activity.

Neonatal RASM cells were transfected with-1575 NPR-A LUC (0.5 μg) by electroporation. Cells were co-transfected with a constitutively active CMV-Renilla luciferase reporter (0.25 μg) to control for differences in transfection efficiency. 24 hrs post-transfection, cells were treated with the vitamin D analogues, or vehicle, as indicated. The incubation was continued for 48 hrs at which point cells were harvested, lysates were generated and luciferase (firefly and Renilla) measurements were made.

Effect of VDRA/Vitamin D Analogs on NPR-A Activity

Cells were preincubated for 48 hrs in 1,25 dihydroxyvitamin D (VD), paricalcitol, HECTOROL (calcitriol) or the activated form of HECTEROL (calcitriol). At that point medium was changed, the nonselective phosphodiesterase inhibitor IBMX (10⁻⁴ M) was added, and the incubation was continued for 10 min at 37 C. ANP (10⁻⁷ M) was then added to each culture and the incubation extended an additional 10 minutes. Medium was then aspirated, cells were lysed with TCA and soluble extracts subjected to ether extraction, neutralization and radioimmunoassay for cGMP levels. All cGMP levels presented here are normalized per μg of soluble protein present in the extract.

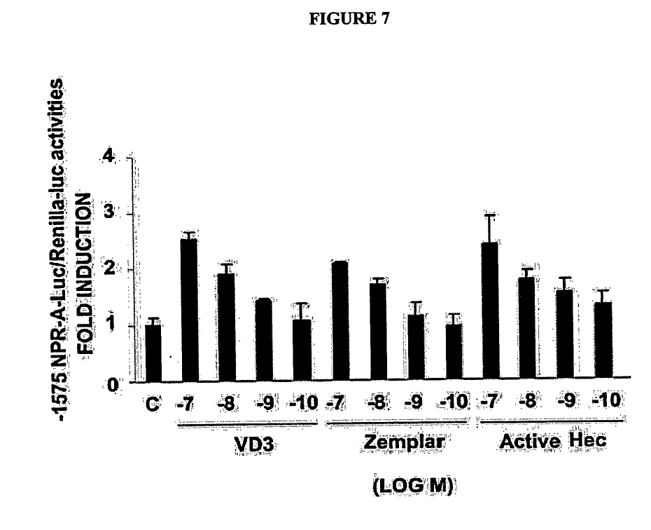

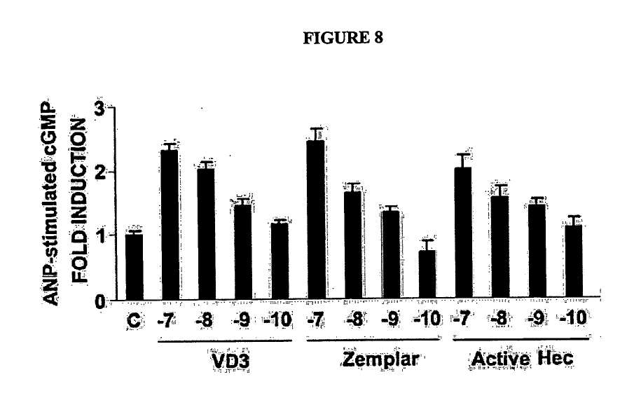

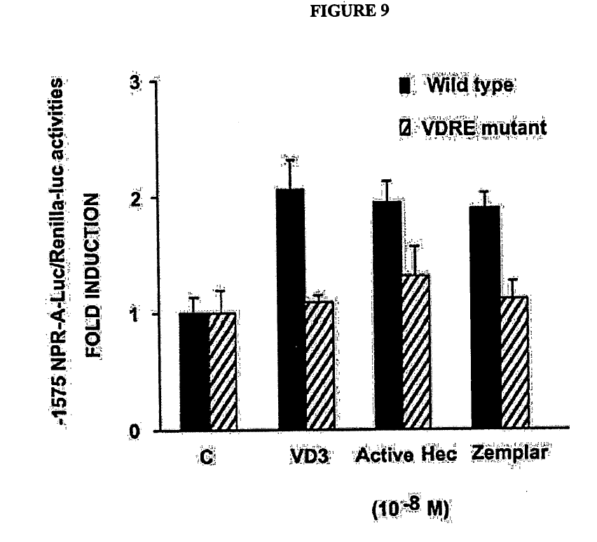

Results are shown in FIGS. 7, 8 and 9.

Effect of Vitamin D Analogues on hBNP Gene Promoter Activity.

Neonatal rat ventricular myocytes were transfected with −1595 hBNP LUC (0.25 μg) by electroporation as described previously. Co-transfected CMV-Renilla luciferase (0.25 μg) was used to normalize samples for differences in transfection efficiency, as described above. In selected cases, expression vectors for the human vitamin D receptor (hVDR) (0.3 μg) and human retinoid X receptor (hRXR) (0.3 μg) were co-transfected with the BNP luciferase reporter. Where indicated samples were treated with endothelin (10⁻⁷ M) or one of the vitamin D analogues.

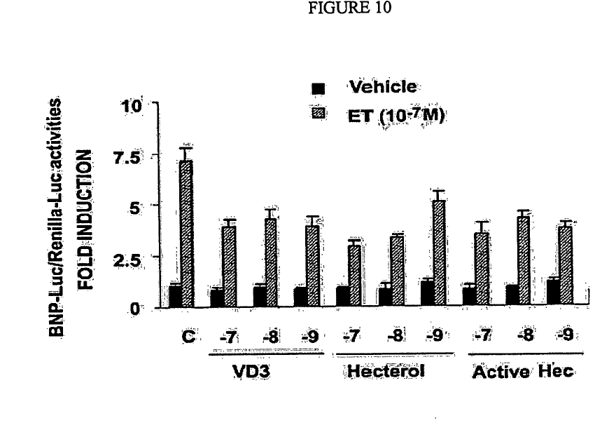

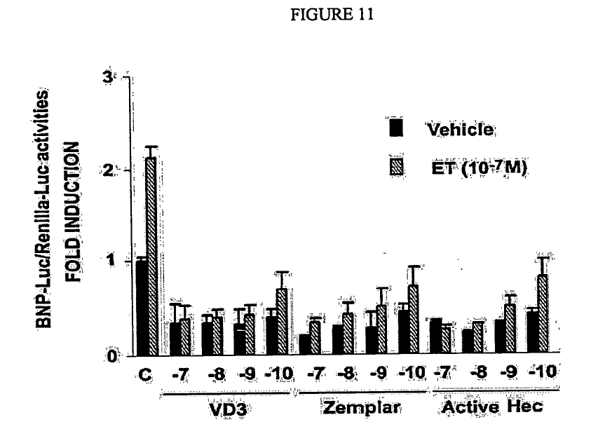

Results are shown in FIGS. 10 and 11.

Measurement of Cdk2 Activity.

Cells were treated with vehicle or the vitamin D analogues for the intervals indicated. Cells were lysed with lysis buffer and 100 μg of supernatant protein was incubated with 1 μg of anti-Cdk2 antibody and 10 μl of protein G-Sepharose for 1-2 hrs at 4 C. Immune complex kinase assays were carried out as described previously using the immunoprecipitates generated above together with 2 μg of histone 1 and γ-³²P-ATP in kinase buffer. Reaction products were separated on denaturing SDS-polyacrylamide gels which were then dried and exposed to X-ray film.

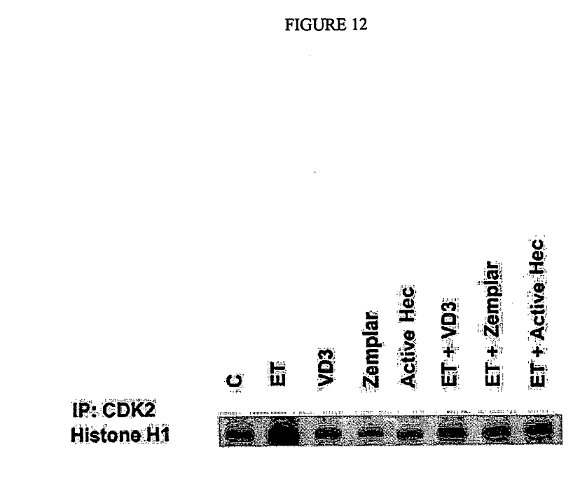

Results are shown in FIG. 12.

The current study indicates that VDRAs possess functional activity in the cardiovascular system that is similar, both qualitatively and quantitatively, to that previously demonstrated for the native hormone, 1,25 dihydroxyvitamin D. Specifically, the major findings of this study indicate that VDRAs: 1) increase activity of the type A natriuretic peptide receptor (NPR-A) in neonatal rat aortic smooth muscle cells, 2) increase NPR-A gene promoter activity in the same cells through a vitamin D response element, 3) suppress ET-dependent stimulation of the BNP gene promoter in cultured neonatal rat ventricular myocytes, 4) inhibit endothelin-dependent stimulation of ³H-thymidine incorporation into DNA and Cdk2 activity in adult rat aortic smooth muscle cells. Collectively, these data suggest that paricalcitol, like 1,25 dihydroxyvitamin D, may possess cardio-protective effects that control hypertrophy of cardiac myocytes in the myocardial wall and vasculo-protective effects that both limit cell proliferation in the remodeling vascular wall and increase the expression/activity of the anti-proliferative, vasorelaxant natriuretic peptide/NPR system in the vasculature.

### EXAMPLE 5

### Vascular Access Changes in Subjects Treated with Zemplar

Methods: A historical cohort of 2112 adult patients new to HD, with an AV fistula as the initial primary vascular access were followed over a 35-month period (Jan 1999 thru Nov 2001) using a dialysis provider database. Patients were treated with Zemplar or no vitamin D therapy; patients receiving Zemplar therapy received at least 10 doses and remained on the same therapy. Descriptive summary statistics were used to summarize baseline characteristics and the total number of vascular access changes per year between treatment modalities. In addition, regression models were used to evaluate the association between Zemplar or no vitamin D therapy and the total number of vascular access changes per year.

Results: The data set contained 577 patients treated with Zemplar and 1535 patients who received no vitamin D therapy. The total number of vascular access changes averaged 0.6 changes per year in Zemplar patients and 0.9 changes per year in no D Patients (p=0.0034). Negative binomial regression was performed to control for baseline covariates; this revealed that the No D group were associated with 28% more vascular access changes than Zemplar patients (p=0.038).

1. A sustained release pharmaceutical composition for preventing, treating and delaying progression of cardiovascular, cerebrovascular and peripheral vascular diseases, especially heart failure, cardiomyopathy, atherosclerosis, myocardial infarction, and cerebrovascular accident, comprising:
a therapeutically effective amount of a VDRA or Vitamin D analog; and optionally
a therapeutically effective amount of at least one member of the group consisting of an angiotensin converting enzyme inhibitor, an angiotensin (11) receptor (I) blocker, and an aldosterone blocker.

2. A sustained release pharmaceutical composition according to claim 1, wherein said VDRA or Vitamin D analog is selected from the group consisting paricalcitol, calcitriol and doxercalciferol.

3. A sustained release pharmaceutical composition according to claim 1 is a transdermal patch.

4. A sustained release pharmaceutical composition according to claim 1 is an oral dosage form.

5. A sustained release pharmaceutical composition according to claim 1 is a subcutaneous dosage form.

6. A sustained release pharmaceutical composition according to claim 1 is an injectable dosage form.

7. A sustained release pharmaceutical composition according to claim 6, wherein said injectable dosage form is a member of the group consisting of a subcutaneous dosage form and a depot dosage form.

8. A sustained release pharmaceutical composition according to claim 5 is an implantable form.

9. A pharmaceutical composition for treating, preventing or delaying progression of vascular disease in a mammal, comprising:
a therapeutically effective amount of Vitamin D receptor activator or Vitamin D analog; and
an optional therapeutically effective amount of at least one member of the group consisting of an angiotensin converting enzyme inhibitor, an angiotensin (II) receptor (I) blocker, and an aldosterone blocker

10. A pharmaceutical composition according to claim 9, wherein said cardiovascular disease is selected from the group consisting of heart failure, cardiomyopathy, atherosclerosis, myocardial infarction, cerebrovascular accident and peripheral vascular disease.

11. A pharmaceutical composition according to claim 9, wherein said Vitamin D or Vitamin D analog is selected from the group consisting of paricalcitol, calcitriol, and doxercalciferol.

12. A pharmaceutical composition according to claim 9 is a transdermal patch.

13. A pharmaceutical composition according to claim 9 is an oral dosage form.

14. A pharmaceutical composition according to claim 9 is a subcutaneous dosage form.

15. A pharmaceutical composition according to claim 9 is an injectable dosage form.

16. A pharmaceutical composition according to claim 15, wherein said injectable dosage form is a member of the group consisting of a subcutaneous dosage form and a depot dosage form.

17. A pharmaceutical composition according to claim 14 is an implantable form.

18. A method of preventing, treating and delaying disease progression of vascular disease in a mammal, comprising the step of administering to said mammal a pharmaceutical composition according to claim 9.

19. A method according to claim 18, wherein the administering step is continuous.

20. A method according to claim 18, wherein the administering step is carried out using a transdermal patch.

21. A method according to claim 18, wherein the administering step is carried out using an oral dosage form.

22. A method according to claim 18, wherein the administering step is carried out using an injectable dosage form.

23. A method according to claim 18, wherein the administering step is carried out using a subcutaneous dosage form.

24. A method of treating, inhibiting or preventing vascular disease in a mammal by reducing PAI-1 expression in said mammal, comprising the step of administering to said mammal an effective amount of a Vitamin D receptor activator or Vitamin D analog.

25. A method according to claim 24, wherein said Vitamin D receptor activator is paricalcitol or calcitriol.

26. A method according to claim 24, wherein said Vitamin D analog is doxercalciferol or alfacalcidol.

27. A method of treating, inhibiting or preventing thrombosis in a mammal in need of said treatment, inhibition or prevention, comprising the step of administering to said mammal an effective amount of a Vitamin D receptor activator or Vitamin D analog.

28. A method according to claim 27, wherein said Vitamin D receptor activator is paricalcitol or calcitriol.

29. A method according to claim 27, wherein said Vitamin D analog is doxercalciferol or alfacalcidol.
